(19) United States
(12) Patent Application Publication
(10) Pub. No.: US 2008/0043802 A1
(43) Pub. Date: Feb. 21, 2008

(54) Methods of determining shape memory coefficients of fabrics

(75) Inventors: Jinlian Hu, Hong Kong (CN); Siu Ping Jane Chung, Hong Kong Sar (CN); Edward Newton, Hong Kong Sar (CN); Yuen Kei Li, Hong Kong Sar (CN)

Correspondence Address: BUCHANAN, INGERSOLL & ROONEY PC, POST OFFICE BOX 1404, ALEXANDRIA, VA, 22313-1404, US

(73) Assignee: The Hong Kong Polytechnic University, Hong Kong SAR (CN)

(21) Appl. No.: 11/485,480

(22) Filed: Jul. 13, 2006

(51) Int. Cl. G01N 25/00; G01N 33/36

(52) U.S. Cl. 374/46; 73/159

(57) ABSTRACT

Shape-memory polymer is a new kind of material utilizing shape memorizing mechanism of elastomer of polyurethane family. These materials exhibit novel properties such as sensing (thermal), actuaton, high damping, adaptive responses, super-elasticity capability and air permeability. Recently, they are used and applied to finish textiles and garments. Although there are some characterization methods for shape memory polymers, there is currently no characterization method to evaluate the effects of shape memory fabrics. This invention provides a method to determine the shape memory coefficient of fabrics. The fabric is first deformed from the original state to a deformed state and then subjected to a switch temperature. Different parameters are measured at the original state before and after the fabric is subjected to the switch temperature to determine the shape memory coefficient.

### FIELD OF THE INVENTION

The present invention relates to the field of shape memory coefficients of materials. In particular, the present invention relates to classification methods of shape memory fabric.

### BACKGROUND OF THE INVENTION

A shape-memory polymer is a material utilizing shape memorizing mechanisms of elastomers of the polyurethane family. These materials exhibit novel properties such as sensing (thermal), actuation, high damping, adaptive responses, super-elasticity capability and air permeability. Recently, shape memory polymers have been applied to finish textiles and garments.

Although there are some characterization methods for shape memory polymers, there is currently no characterization method to evaluate the effects of shape memory fabrics. GB 885035 to Cluett Reabody & Co Inc discloses a wrinkle measuring device using photo-electronic evaluation for wrinkle images. Later, the evaluation concept was further modified to become a wrinkle recovery test apparatus in U.S. Pat. No. 3,094,866 to Weller et al.

### OBJECTS OF THE INVENTION

Therefore, it is an object of this invention to provide a method for evaluating the shape memory properties of a fabric, and/or to substantially ameliorate at least one or more of the problems associated with the prior art. As a minimum, it is an object of this invention to provide the public with a useful choice.

### SUMMARY OF THE INVENTION

Accordingly, this invention provides a method of determining a shape memory coefficient of a fabric. The fabric has an original state at an original temperature to be deformed to a deformed state. The method of this invention includes the steps of:
measuring a first parameter of the fabric at the original state;
deforming the fabric from the original state to the deformed state;
subjecting the fabric to the switch temperate;
measuring a second parameter of the fabric after the fabric is subjected to the elevated temperature; and
comparing the first and second parameters to determine the shape memory coefficient of the fabric.

Preferably, the first and second parameters are selected from the group consisting of length, arm wrinkle recovery angle, crease recovery angle, flat appearance, crease retention, dimension changes, tensile strength, tearing strength, abrasion resistance, air permeability, and their mixtures thereof.

The shape memory coefficient of the fabric can be determined by the formula

$$S = 1 - \frac{\vert a - b \vert}{a} ,$$

wherein S is the shape memory coefficient, a is the first parameter, and b is the second parameter.

### DETAILED DESCRIPTION OF THE PREFERRED EMBODIMENT

This invention is now described by way of example in the following paragraphs.

Objects, features, and aspects of the present invention are disclosed in or are apparent from the following description. It is to be understood by one of ordinary skilled in the art that the present discussion is a description of exemplary embodiments only, and is not intended as limiting the broader aspects of the present invention, which broader aspects are embodied in the exemplary constructions.

The purpose of this invention is to develop a characterization method for shape memory fabrics so that the degree or dimension of shape recovery of shape memory fabrics be evaluated. The evaluation methods may utilize all existing methods for evaluating fabrics properties including length, area, flat appearance, crease retention, bagging recovery, and recovery angle, and any other suitable methods.

As generally known, “shape memory fabric” refers to a fabric capable of being deformed from an original state at an original temperature to a deformed state, and then reverted back to the original state when the fabric is subjected to a switch temperature.

The method of this invention includes the steps of:
i) Measuring a first parameter of the fabric at the original state. The first parameter refers to any one of the above fabric properties.
ii) Deforming the fabric from the original state to the deformed state.
iii) Subjecting the deformed fabric to the switch temperature, so that the fabric can be revered to the original state if it is a shape memory fabric.
iv) Measuring a second parameter of the fabric after it is subjected to the switch temperature. Of course, the second parameter shall correspond with the first parameter referring to a same property.
v) Comparing the first and second parameters to determine the shape memory coefficient of the fabric.

Often, the original temperature is room temperature, and the switch temperature is usually higher than room temperature, typically at about 40 to 60° C. Therefore, it may be necessary to heat the fabric to reach the switch temperature.

i) Measuring a First Parameter of the Fabric at the Original State

This includes the measurement of the first parameter, including length, area, height, flat appearance, crease retention, bagging recovery, and/or recovery angle. The measurements of such parameters are well known and will not be further illustrated here.

ii) Deforming the Fabric from the Original State to the Recovery State

In order to obtain comparable results, it is preferred to deform the fabric using repeatable procedures. Several different methods may be used as follows:

(a) Using known weights to create wrinkle, flatten crease, or create crease line.

(b) In the evaluation of bagging recovery, the Instron machine is used for making a bag from a flat fabric.

iii) Subjecting the Deformed Fabric to the Switch Temperature

Again, it is preferred to revert the fabric from the recovery state to the original state using repeatable procedures. Suitable methods may include:

(a) Using a water tank with electronic heater device so that the temperature of water can be controlled.

(b) Using a controllable heater or steam generator.

(c) Using washing and tumble dry machines with temperature regulator

iv) Measuring a Second Parameter of the Fabric after it is Subjected to the Switch Temperature

This step is relatively simple, mainly repeating the process in step i) after step iii).

v) Comparing the First and Second Parameters to Determine the Shape Memory Coefficient of the Fabric.

The first and second parameters can then be compared to determine the shape memory coefficient of the fabric. The shape memory coefficient of the fabric may be determined by the formula

$$S = 1 - \frac{\vert a - b \vert}{a} ,$$

wherein S is the shape memory coefficient, a is the first parameter, and b is the second parameter. The shape memory “property” of the fabric is good if S is close to 1, which means that the fabric can restore its original state more closely.

### EXAMPLES

### Example 1

### Objective Evaluation: Shape Memory Coefficient (Flat Fabric)

### Make a Crease

3 warp and 3 weft specimen, each 4 cm×1.5 cm were cut from shape memory fabrics
3 warp and 3 weft specimen, each 4 cm×1.5 cm were cut from untreated fabrics.

### Draw a crease line in the middle of each specimen.

### Fold each specimen with a foil plate to make a crease in the middle of specimen

### Use the foil plate to push the specimen into a plastic holder to keep the crease in position, and then remove the foil.

### Measure Wrinkle Angle at Original State

### Measure the wrinkle angles of the specimens using commercial available wrinkle recovery tester.

### Deform the Specimen

### Use a weight to press the entire specimen for one hour.

### Subjecting the Deformed Fabric to the Switch Temperature

### Turn on the power of the thermo-regulator in the water tank to increase the water temperature, and regulate the water temperature at, for example, 40° C.

### Put the specimens into the heated water at the switch temperature (e.g. 40° C.) for sufficient time to allow the fabric specimens to return to the original if it is indeed a shape memory fabric.

### Hang the specimens onto the drying frame and dry the specimens.

### Measure the Wrinkle Angles after being Subjected to the Switch Temperature

Measure the wrinkle angles of the specimens after being subjected to the switch temperature using commercial available wrinkle recovery tester. The shape memory coefficient can then be determined.

### Example 2

### Objective Evaluation: Shape Memory Coefficient (Crease Fabric)

### Cut Fabrics from a Crease Position

### Prepare crease during finishing in shape memory and untreated fabrics.

### Cut fabrics with a crease in the middle.

3 pieces of 4 cm×1.5 cm fabric stripes were cut from shape memory fabrics.
3 pieces of 4 cm×1.5 cm fabric stripes were cut from untreated fabrics.

### Measure the Crease Recovery Angles at Original State

### Measure the crease recovery angles at original state (O_{c}) using commercially available crease recovery tester.

### Deform the Specimen

### Use a weight to press the entire specimen flat for 30 minutes,

### Subjecting the Deformed Fabric to the Switch Temperature

### Turn on the power of the thermo-regulator in the water tank to increase the water temperature, and regulate the water temperature at, for example, 40° C.

### Put the specimens into the heated water at the switch temperature (e.g. 40° C.) for sufficient time to allow the fabric specimens to return to the original if it is indeed a shape memory fabric.

### Hang the specimens onto the drying frame and dry the specimens.

### Measure the Crease Angles after Recovery

Measure the crease recovery angles of the specimens after being subjected to the switch temperature using commercial available wrinkle recovery tester. The shape memory coefficient can then be determined.

### Additional Parameters that can be Measured

### Other than wrinkle angle and crease angle, the following parameters may also be measured according to their respective standards.

### Appearance Test:

### Flat appearance (AATCC Test Method 124-2001): measure the grading for the degree of wrinkles on the fabrics after washing.

### Crease Retention (AATCC Test Method 88C-2003): measure the grading for the degree of crease retention on the fabrics after washing.

### Dimension Test

### Dimension Changes (AATCC Test Method 135-2001): measure the length and width of fabrics after washing.

### Durability and Mechanical Properties:

### Tensile Strength (ASTM Designation: D 5034-95): measure the breaking force and elongation of fabric.

### Tearing Strength (ASTM Designation: D 1424-96): measure the force required to propagate a single-rip tear starting from a cut in a fabric.

### Abrasion resistance (BS EN ISO 12947-1:1999): measure the number of rubs that the fabrics can withstand.

### Comfort Test:

### Air permeability (BS EN ISO 9237:1995): measure the velocity of airflow passing through the fabrics.

It should be noted that although each and every one of the above parameters can be used, a plurality of the above parameters can also be used simultaneously or independently to determine the shape memory coefficient.

The method of this invention provides objective means to evaluate the shape memory properties of a fabric

While the preferred embodiment of the present invention has been described in detail by the examples, it is apparent that modifications and adaptations of the present invention will occur to those skilled in the art. It will be apparent that such modifications and adaptation will be understood by those skilled in the art to fall within the scope of the present invention without departing from the general inventive concept. Furthermore, the embodiments of the present invention shall not be interpreted to be restricted by the examples or figures only. It is to be expressly understood, however, that such modifications and adaptations are within the scope of the present invention, as set forth in the following claims. For instance, features illustrated or described as part of one embodiment can be used on another embodiment to yield a still further embodiment. Thus, it is intended that the present invention cover such modifications and variations as come within the scope of the claims and their equivalents.

1. A method of determining a shape memory coefficient of a fabric, said fabric having an original state at an original temperature and to be deformed to a deformed state, said method including the steps of:
measuring a first parameter of the fabric at the original state;
deforming the fabric from the original state to the deformed state;
subjecting the fabric to a switch temperate;
measuring a second parameter of the fabric after the fabric is subjected to the switch temperature; and
comparing the first and second parameters to determine the shape memory coefficient of the fabric.

2. The method of claim 1, wherein the first and second parameters are selected from the group consisting of length, area, wrinkle recovery angle, crease recovery angle, flat appearance, crease retention, dimension changes, tensile strength, tearing strength, abrasion resistance, air permeability, and combinations thereof.

3. The method of claim 2, wherein the first and second parameters are the lengths of the fabric at the original and the deformed states respectively.

4. The method of claim 2, wherein the first and second parameters are the areas of the fabric at the original and the deformed states respectively.

5. The method of claim 2, wherein the first and second parameters are the wrinkle recovery angles of the fabric at the original and the deformed states respectively.

6. The method of claim 2, wherein the first and second parameters are the crease recovery angles of the fabric the original and the deformed states respectively.

7. The method of claim 1, wherein the shape memory coefficient of the fabric is determined by the formula
$$S = 1 - \frac{\vert a - b \vert}{a} ,$$
wherein S is the shape memory coefficient, a is the first parameter, and b is the second parameter.
